(19) United States
(12) Patent Application Publication
(10) Pub. No.: US 2025/0312276 A1
(43) Pub. Date: Oct. 9, 2025

(54) Oil-in-water emulsion comprising a semiochemical composition, suitable for administration in the form of a milk

(71) Applicant: SIGNS, APT (FR)

(72) Inventors: Maréva REBOUL, APT (FR); Mélinda TEP, APT (FR); Patrick PAGEAT, APT (FR)

(21) Appl. No.: 19/034,916

(22) Filed: Jan. 23, 2025

(30) Foreign Application Priority Data
Apr. 5, 2024 (FR) 2403565

(51) Int. Cl. A61K 9/107; A61K 31/19; A61K 31/22; A61K 31/225; A61K 47/10; A61K 47/14; A61K 47/26; A61K 47/36; A61K 47/44

(52) U.S. Cl. CPC A61K 9/107; A61K 31/19; A61K 31/22; A61K 31/225; A61K 47/10; A61K 47/14; A61K 47/26; A61K 47/36; A61K 47/44

(57) ABSTRACT

The invention relates to the field of semiochemical products, and describes in particular a composition in the form of an oil-in-water emulsion comprising an aqueous phase and an oily phase comprising a semiochemical composition, particularly suitable for being administered in particular in the form of a milk, as well as its preparation process. The invention also relates to the non-therapeutic use of the composition according to the invention for topical, in particular cutaneous, administration to an animal or human.

### TECHNICAL FIELD OF THE INVENTION

The invention relates to the field of semiochemicals, and describes in particular a composition in the form of an oil-in-water emulsion comprising an aqueous phase, and an oily phase comprising a semiochemical composition, in particular suitable for administration especially in the form of a milk, as well as its preparation process. The invention also relates to the non-therapeutic use of the composition according to the invention for topical, in particular cutaneous, administration to an animal or human.

### PRIOR STATE OF THE ART

Semiochemicals according to the invention correspond to mixtures of several chemical compounds, described for the first time in particular by Silverstein, R. M. (1977). Semiochemicals are chemical substances released by a plant or animal that provoke a non-therapeutic behavioural or physiological response in another organism. When the semiochemical affects an individual of the same species (“intra-specific semiochemical”), it is called a pheromone (Wyatt T. D., 2003). There are several types of pheromones that enable different types of behavioural interaction or communication, such as soothing pheromones, sex pheromones, territorial marking pheromones and warning pheromones. When the semiochemical affects an individual of a different species (“interspecific semiochemical”), it is called an allelochemical substance. Among allelochemical substances, an allomone is an allelochemical that triggers a reaction in the target species, this reaction being favourable to the species that releases the allomone (the opposite is a kairomone).

Since 1995, specialities whose active ingredients are semiochemical agents have been marketed. Some of their uses require the product to be deposited on the animal's skin, for example to obtain the desired effects on an animal in an open environment or to protect it from pests such as parasites or biting insects, or on human skin. The physico-chemical constraints of semiochemical components, and in particular their volatility and insolubility in water, have led to the use of excipients such as ethoxydiglycol in marketed specialities. The use of this type of excipient is now increasingly restricted by current regulations, but also by the general public, who tend to reject them because of the way they are synthesised and their potential direct and environmental toxicity. It therefore appeared necessary to develop aqueous formulations of semiochemical compounds that were non-toxic and environmentally friendly, and in particular complied with EC Regulation No. 1223/2009 of the European Parliament and Council of 30 Nov. 2009 on cosmetic products.

### DISCLOSURE OF THE INVENTION

The aim of the invention is to develop a composition based on semiochemical products (also referred to as compounds, active ingredients or agents) in a new dosage form, preferably non-toxic and environmentally friendly, in particular in the form of an oil-in-water (O/W) emulsion, preferably in the form of a stable and homogeneous oil-in-water (O/W) emulsion, for topical administration, in particular cutaneous, to an animal, in particular a mammal or a human, or for topical administration, in particular cutaneous, to farm animals treated as a group, in particular to prevent invertebrates (haematophagous or not) from settling on the skin, or to prevent bites by haematophagous invertebrates, in particular haematophagous insects, such as mosquitoes, mites or ticks; or to prevent infestation by parasites such as poultry red mite (Dermanyssus sp. or Ornithonyssus sylviarum). Preferably, the topical administration of the composition according to the invention is carried out by uniform deposition on one or more body areas, in particular on all the body areas treated, for example in the form of a continuous layer (uniformly and completely covering the treated body area).

### SUMMARY OF THE INVENTION

The invention is defined by the independent claims. The dependent claims define particular or preferred embodiments of the invention.

According to a first aspect, the invention relates to a composition in the form of an oil-in-water (O/W) emulsion (referred to as an O/W composition or composition according to the invention) comprising:
a) an aqueous phase comprising water, at least one humectant chosen from polyols and sugars, and at least one gelling agent chosen from polysaccharides,
b) an oily phase comprising at least one emollient agent, at least one emulsifying agent, and a semiochemical composition,
wherein said semiochemical composition comprises (i) 3-cyclopentyl propionic acid and 3-cyclohexyl propionic acid, or (ii) bis(2-ethylhexyl) adipate and 1-isopropyl-2,2-dimethyltrimethylene diisobutyrate.

According to a second aspect, the invention relates to a process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent, said semiochemical composition and optionally said at least one additive are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, a perfume is added;
f) the composition is recovered in the form of the oil-in-water (O/W) emulsion, particularly stable and homogeneous, obtained in step c), d) or e).

According to a third aspect, the invention relates to the non-therapeutic use of a composition according to the invention for topical, in particular cutaneous, administration to an animal or a human, in particular on the skin of an animal or a human, in particular to prevent invertebrates (haematophagous or not) from settling on the skin, or to prevent bites by haematophagous invertebrates, in particular haematophagous insects, such as mosquitoes, mites or ticks; or to prevent infestation by parasites such as poultry red mite (Dermanyssus sp. or Ornithonyssus sylviarum).

### DETAILED DESCRIPTION OF THE INVENTION

The invention is now described in more detail and in a non-limiting way in the following description. Unless otherwise stated, all percentages relating to quantities are percentages by mass. Any reference to ranges of values in the description and/or claims implies, unless otherwise stated, that the limits of the ranges are included.

The invention relates to a composition in the form of an oil-in-water (O/W) emulsion comprising:
a) an aqueous phase comprising water, at least one humectant chosen from polyols and sugars, and at least one gelling agent chosen from polysaccharides,
b) an oily phase comprising at least one emollient agent, at least one emulsifying agent, and a semiochemical composition,
wherein said semiochemical composition comprises (i) 3-cyclopentyl propionic acid and 3-cyclohexyl propionic acid, or (ii) bis(2-ethylhexyl) adipate and 1-isopropyl-2,2-dimethyltrimethylene diisobutyrate.

In a particular embodiment of the invention, said semiochemical composition comprises (i) from 40% to 60% by weight of 3-cyclopentyl propionic acid and from 40% to 60% by weight of 3-cyclohexyl propionic acid, or (ii) from 40% to 60% by weight of bis(2-ethylhexyl) adipate, and from 40% to 60% by weight of 1-isopropyl-2,2-dimethyltrimethylene diisobutyrate, the total sum of the compounds present in said semiochemical composition being equal to 100%.

In a preferred embodiment of the invention, said semiochemical composition comprises (i) 50% by weight of 3-cyclopentyl propionic acid and 50% by weight of 3-cyclohexyl propionic acid, or (ii) 50% by weight of bis(2-ethylhexyl) adipate and 50% by weight of 1-isopropyl-2,2-dimethyltrimethylene diisobutyrate.

### Galenics

The composition according to the invention is in the form of an oil-in-water (O/W) emulsion, in particular an oil-in-water emulsion which is stable and has a homogeneous appearance, particularly to the naked eye. In particular, the composition is in the form of a milk, preferably in the form of a white milk with a thick, opaque texture.

In the context of the invention, the term “emulsion” refers to a mixture of two immiscible liquid substances, such as oil and water. In an oil-in-water (O/W) emulsion, the oil is dispersed in the water in the form of droplets, in particular small droplets.

In the context of the invention, the term “stable emulsion” refers to an emulsion showing no signs of instability (phase separation, increase in droplet size, etc.), in particular an emulsion which does not release water or oil and which remains in its initial form over time and under different temperature conditions, for example during a 3 months storage at a temperature of between 5° C. and 40° C.

In the context of the invention, the term “homogeneous emulsion” refers to an emulsion whose surface is smooth and uniform to the naked eye.

### Aqueous Phase

The composition according to the invention comprises an aqueous phase, in particular a continuous aqueous phase (also known as the external phase).

The aqueous phase of the composition according to the invention comprises water, preferably demineralised water or purified water, and optionally one or more water-miscible or at least partly water-miscible compounds, for example a skin care agent such as ALOE VERA, a hydrolat, urea or an antistatic agent.

In the context of the invention, the term “skin care agent” refers to an agent for maintaining the skin in good condition, e.g., healthy-looking.

In the context of the invention, the term “antistatic agent” refers to an agent that reduces static electricity by neutralising the electrical charge on a surface.

In a particular embodiment of the invention, the composition according to the invention comprises from 60% to 95% by weight of water, preferably from 70% to 80% by weight of water, relative to the total weight of the composition.

In a preferred embodiment of the invention, the composition according to the invention comprises from 62% to 91% by weight of water, preferably from 73.0% to 76.5% by weight of water, even more preferably 74.4% (milk for application to an adult) or 76.1% (milk for application to a baby) by weight of water, relative to the total weight of the composition.

The aqueous phase of the composition according to the invention may comprise water, at least one humectant chosen from polyols and sugars, and at least one gelling agent chosen from polysaccharides.

### Humectant

According to a particular embodiment, the aqueous phase of the composition according to the invention also comprises at least one humectant chosen from polyols and sugars. In particular, the aqueous phase of the composition according to the invention also comprises a single humectant chosen from polyols or sugars, preferably polyols. Said at least one humectant is notably soluble in the aqueous phase.

In the context of the invention, the term “humectant” (also called moisturising agent) refers to a compound or mixture of compounds that retain water due to their hygroscopic properties.

In the context of the invention, the term “polyol” refers to any organic compound comprising at least two free hydroxyl groups (—OH), such as glycols.

In the context of the invention, the term “sugar” refers to simple carbohydrates, in particular any organic molecule consisting of one ose (monosaccharide) or two oses (disaccharide).

In a particular embodiment of the invention, said at least one humectant is chosen from polyols selected from the group consisting of glycerol (also called glycerin), propylene glycol, polyethylene glycols (PEG), xylitol, isomalt, maltitol, mannitol, polydextrose, sorbitol, isomaltitol, lactitol, and mixtures thereof; and/or said at least one humectant is chosen from sugars selected from the group consisting of fructose, glucose, lactose and mixtures thereof. In a preferred embodiment of the invention, said at least one humectant is a polyol, in particular glycerol.

Said at least one humectant, i.e. one or more humectants, preferably one humectant, may be present in the composition in an amount ranging from 4% to 12% or 4.5% to 12% by weight, preferably from 4% to 6% by weight, relative to the total weight of the composition.

Preferably, said at least one humectant is glycerol and is present in the composition in an amount ranging from 4.5% to 12% by weight, preferably 5% by weight, relative to the total weight of the composition.

### Gelling Agent

In a particular embodiment, the aqueous phase of the composition according to the invention also comprises at least one gelling agent chosen from polysaccharides. In particular, the aqueous phase of the composition according to the invention also comprises a single gelling agent chosen from polysaccharides. Said at least one gelling agent is notably soluble in the aqueous phase. Said at least one gelling agent is a hydrophilic gelling agent.

In the context of the invention, the term “gelling agent” refers to an agent capable of increasing the viscosity of the oil-in-water emulsion and/or allowing the various constituents of the composition to adhere together.

In the context of the invention, the term “polysaccharides” (also known as glycans, polyosides or polyholosides) refers to complex carbohydrates formed by the polymerisation of oses (monosaccharides) linked together by glycosidic bonds.

In a particular embodiment of the invention, the polysaccharides are chosen from the group consisting of xanthan gum, guar gum, cellulose, cellulose derivatives, maltodextrin, maltodextrin derivatives and mixtures thereof, preferably xanthan gum.

The polysaccharides may be present in the composition in an amount ranging from 0.05% to 1% or 0.05% to 0.9% by weight, preferably from 0.1% to 0.3% by weight, relative to the total weight of the composition.

Preferably, the polysaccharides are xanthan gum and are present in the composition in an amount ranging from 0.05% to 0.9% by weight, preferably from 0.2% to 0.4% by weight, even more preferably 0.2% by weight, relative to the total weight of the composition.

In a particular embodiment of the invention, the aqueous phase of the composition comprises glycerol in an amount ranging from 4.5% to 12% by weight, preferably 5% by weight, and xanthan gum in an amount ranging from 0.05% to 0.9% by weight, preferably from 0.2% to 0.4% by weight, even more preferably 0.2% by weight, all the percentages being given relative to the total weight of the composition.

### Oily Phase

The composition according to the invention comprises an oily phase, in particular a dispersed oily phase (also called discontinuous or internal phase).

The oily phase of the composition according to the invention may comprise at least one emollient agent, at least one emulsifying agent and a semiochemical composition. In particular, said at least one emollient agent, said at least one emulsifying agent and said semiochemical composition are soluble in the oily phase.

The oily phase may be present in an amount ranging from 3% to 30% by weight, preferably 15% to 25% by weight, based on the total weight of the composition.

### Emollient Agent

According to a particular embodiment, the oily phase of the composition according to the invention also comprises at least one emollient agent, i.e. one or more emollient agent(s), preferably at least two emollient agents. Said at least one emollient agent is notably soluble in the oily phase.

In the context of the invention, the term “emollient agent” refers to an agent to soften the skin and increase its suppleness during topical, in particular cutaneous, administration of the composition according to the invention.

In a particular embodiment of the invention, said at least one emollient agent is selected from the group consisting of hydrogenated or non-hydrogenated vegetable oils, in particular vegetable oils comprising triglycerides of unsaturated fatty acids (e.g., olive oil, rapeseed oil, sunflower oil, sesame oil, linseed oil, hemp oil, walnut oil, hazelnut oil, coconut oil, argan oil, jojoba oil, sweet almond oil, shea butter, castor oil, avocado oil, apricot kernel oil, macadamia oil, . . . ), silicone oils (e.g., dimethicone, . . . ), mineral oils and waxes (e.g., Parrafine, . . . ), fatty alcohols (e.g., arachidyl alcohol, batyl alcohol, behenyl alcohol, C₁₂-C₁₃ alcohols, cetearyl alcohol, cetyl alcohol, coconut alcohol, isocetyl alcohol, octyldodecanol, palm alcohol, . . . ), fatty acids and fatty acid esters (e.g., batyl stearate, cetearyl behenate, cetearyl isononanoate, cetyl esters, coconut acid, ethyl linoleate, ethyl linolenate, ethyl olivate, ethylhexyl cocoate, ethylhexyl myristate, glyceryl caprylate, glyceryl laurate/oleate, glyceryl ricinoleate, isocetyl salicylate, isostearyl alcohol, linoleic acid, linolenic acid, oleic acid, palm acid, palmitic acid, wheat germ acid, . . . ), glyceride derivatives (e.g., triglycerides of capric/caprylic acid, cocoglycerides, palm glycerides, . . . ), glycerol derivatives (e.g., glycerol trioctanoate: Tricaprylin, Glycerol tristearate: Tristearin . . . ), and mixtures thereof; preferably said at least one emollient agent is a mixture of emollient agents consisting of glyceride derivatives such as fatty acid triglycerides, and vegetable oils comprising unsaturated fatty acid triglycerides.

Said at least one emollient agent, i.e., one or more emollient agent(s), preferably a mixture of two emollient agents, is present in an amount ranging from 1% to 19% by weight, preferably from 4% to 6% by weight, relative to the total weight of the composition.

In a preferred embodiment of the invention, said at least one emollient is a fatty acid triglyceride, in particular a caprylic/capric acid triglyceride such as that marketed in particular by BASF under the name Myritol 318®, and is present in the composition in an amount ranging from 0.5% to 8% by weight, preferably 3% by weight, relative to the total weight of the composition.

In another preferred embodiment of the invention, said at least one emollient agent is a vegetable oil comprising triglycerides of unsaturated fatty acids, in particular sunflower oil, and is present in the composition in an amount ranging from 1% to 10.2% by weight, preferably 2% by weight, relative to the total weight of the composition.

In another even more preferred embodiment of the invention, said at least one emollient agent is a mixture of emollient agents consisting of glyceride derivatives such as fatty acid triglycerides, in particular a caprylic/capric acid triglyceride such as that marketed in particular by BASF under the name Myritol 318®, and vegetable oils comprising triglycerides of unsaturated fatty acids, in particular sunflower oil, this mixture being present in the composition in an amount ranging from 1% to 19% by weight, preferably from 4% to 6% by weight, even more preferably 5% by weight, relative to the total weight of the composition.

In a particular embodiment of the invention, the aqueous phase of the composition comprises glycerol in an amount ranging from 4.5% to 12% by weight, preferably 5% by weight, and xanthan gum in an amount ranging from 0.05% to 0.9% by weight, preferably from 0.2% to 0.4% by weight, even more preferably of 0.2% by weight, and the oily phase of the composition comprises a mixture of emollient agents consisting of a caprylic/capric acid triglyceride such as Myritol 318® in an amount ranging from 0,5% to 8% by weight, preferably 3% by weight, and a vegetable oil comprising triglycerides of unsaturated fatty acids, such as sunflower oil, in an amount ranging from 1% to 10.2% by weight, preferably 2% by weight, relative to the total weight of the composition, all the percentages being given relative to the total weight of the composition.

### Emulsifying agent

The oily phase of the composition according to the invention also comprises at least one emulsifying agent, preferably one or more emulsifying agent(s). Said at least one emulsifying agent is notably soluble in the oily phase.

Preferably, said at least one emulsifying agent is environmentally friendly, in particular complying with EC regulation N° 1223/2009 of the European Parliament and Council dated 30 Nov. 2009 relating to cosmetic products. The person skilled in the art will therefore be able to choose an environmentally-friendly emulsifying agent on the basis of this EC regulation N° 1223/2009.

In the context of the invention, the term “emulsifying agent” (also called emulsifier) refers to a small molecule composed of a hydrophilic head soluble in the aqueous phase and a lipophilic tail with an affinity for the oily phase. Said at least one emulsifying agent enables the formation of an interfacial film around the droplets in the dispersed phase, thus forming the emulsion and providing stability to the formed emulsion.

Said at least one emulsifying agent may be chosen from non-ionic or ionic (anionic, cationic) surfactants, preferably non-ionic surfactants.

In the context of the invention, the term “surfactant” refers to a compound which reduces surface tension and promotes uniform distribution of the composition according to the invention when used.

In a particular embodiment of this invention, said at least one non-ionic surfactant has a hydrophilic-lipophilic balance (HLB) of between 8 and 20 at 25° C.

In a particular embodiment of this invention, said at least one emulsifying agent is an ionic surfactant, preferably an anionic surfactant chosen from sulphated castor oil, sodium dodecyl sulphate (SDS), sodium coco sulphate (INCI name: Sodium coco sulfate (SCS)), sodium cocoyl isethionate (INCI name: Sodium cocoyl isethionate (SCI)), sodium lauryl sulphoacetate (INCI name: Sodium lauryl sulfoacetate (SLSA)), sodium laureth sulfate (INCI name: Sodium laureth sulphate (SLES)) and sodium lauroyl sarcosinate (INCI name: Sodium Lauroyl Sarcosinate), or a cationic surfactant chosen from cetearyl alcohol (INCI name: Cetearyl Alcohol), behentrimonium methosulphate (INCI name: Behentrimonium Methosulfate (BTMS)), brassica alcohol (and) brassicycle valinate esylate (INCI name: Brassica Alcohol (and) Brassicyl Valinate Esylate), benzalkonium chloride (BAC), didecyldimethylammonium chloride (DDAC), brassicamidopropyl dimethylamine (INCI name: Brassicamidopropyl dimethylamine), Polyquaternium-7, Polyquaternium-10, Polyquaternium-22 and Polyquaternium-37.

In a preferred embodiment of this invention, said at least one emulsifying agent is a non-ionic surfactant, preferably a non-ionic surfactant having an HLB of between 8 and 20 at 25° C., and is selected in particular from the group consisting of alkyl polyglucosides, mixtures of at least one alkyl polyglucoside and at least one fatty alcohol, fatty acid esters, and fatty acid alcohol esters.

Examples of alkyl polyglucosides (APGs) are known to those skilled in the art.

Mixtures of at least one alkyl polyglucoside and at least one fatty alcohol include in particular at least one C₁₂-C₂₀ alkyl polyglucoside and at least one C₁₄-C₂₂ fatty alcohol such as Montanov L® in particular marketed by the company SEPPIC, a mixture of myristic alcohol and myristylic glucoside such as Montanov 14® in particular marketed by the company SEPPIC.

Examples of fatty acid esters include glyceryl stearate, in particular marketed under the name SustOleo GMS® by INOLEX.

Examples of fatty acid alcohol esters include glycol palmitate, in particular marketed under the name Lanol P ecailles® by SEPPIC.

In a particular embodiment of the invention, said at least one emulsifying agent, i.e., one or more emulsifying agent(s), is present in the composition in an amount ranging from 2% to 30% by weight, preferably from 10% to 12% by weight, relative to the total weight of the composition. In a preferred embodiment of the invention, the composition according to the invention comprises several emulsifying agents, in particular a mixture of emulsifying agents consisting of alkyl polyglucosides, mixtures of at least one alkyl polyglucoside and at least one fatty alcohol, fatty acid esters, and fatty alcohol esters, preferably a mixture of emulsifying agents consisting of mixtures of at least one alkyl polyglucoside and at least one fatty alcohol, fatty acid esters and fatty acid alcohol esters.

Preferably, said emulsifying agents are a C₁₂-C₂₀ alkyl polyglucoside and at least one C₁₄-C₂₂ fatty alcohol such as Montanov L® marketed in particular by SEPPIC, a mixture of myristic alcohol and myristylic glucoside such as Montanov 14® marketed in particular by SEPPIC, glyceryl stearate, in particular marketed under the name SustOleoGMS® by INOLEX, and glycol palmitate, in particular marketed under the name Lanol P ecailles® by SEPPIC, and said emulsifying agents are present in the composition in an amount ranging from 2.2% to 26.9% by weight, preferably 11.5% by weight, relative to the total weight of the composition. In particular, Montanov L® is present in the composition in an amount ranging from 1.3% to 8.5% by weight, preferably 4.5% by weight, relative to the total weight of the composition; and/or Montanov 14® is present in the composition in an amount ranging from 0.2% to 5.2% by weight, preferably 1% by weight, relative to the total weight of the composition; and/or SustOleo GMS® is present in the composition in an amount ranging from 0.2% to 5.2% by weight, preferably 2% by weight, relative to the total weight of the composition; and/or Lanol P ecailles® is present in the composition in an amount ranging from 0.5% to 8.0% by weight, preferably 4.0% by weight, relative to the total weight of the composition.

In a particular embodiment of the invention, the aqueous phase of the composition comprises glycerol in an amount ranging from 4.5% to 12% by weight, preferably 5% by weight, and xanthan gum in an amount ranging from 0.05% to 0.9% by weight, preferably from 0.2% to 0.4% by weight, even more preferably 0.2% by weight, and the oily phase of the composition comprises a mixture of emollients consisting of a caprylic/capric acid triglyceride such as Myritol 318® in an amount ranging from 0.5% to 8% by weight, preferably 3% by weight, and a vegetable oil comprising unsaturated fatty acid triglycerides such as sunflower oil in an amount ranging from 1% to 10.2% by weight, preferably 2% by weight, and a mixture of emulsifying agents consisting of Montanov L® present in the composition in an amount ranging from 1.3% to 8.5% by weight, preferably 4.5% by weight, Montanov 14® in an amount ranging from 0.2% to 5.2% by weight, preferably 1% by weight, SustOleo GMS® in an amount ranging from 0.2% to 5.2% by weight, preferably 2% by weight and Lanol P ecailles® in an amount ranging from 0.5% to 8.0% by weight, preferably 4.0% by weight, all the percentages being given relative to the total weight of the composition.

### Semiochemical composition

The oily phase of the composition according to the invention also comprises a semiochemical composition as an active agent, as defined above. Said semiochemical composition is notably soluble in the oily phase described herein. Particular semiochemical compositions are described above and in the preferred embodiments and experimental examples that follow.

The semiochemical composition contained in the composition according to the invention is intended in particular for non-therapeutic use, for example use for their protective effect against pests (parasites, insects, etc.) on the animals or humans to which they are administered or on animals interacting with them, in the form of the O/W composition.

The semiochemical composition may be present in the composition in the form of an oil-in-water emulsion in an amount ranging from 0.5% to 5% by weight, preferably from 1% to 2% by weight of said semiochemical composition, relative to the total weight of the composition according to the invention.

### Additives

The composition according to the invention may also comprise at least one additive provided that it does not interfere with the desired properties of the composition, in particular with its property of being an O/W emulsion.

In a particular embodiment of the invention, said at least one additive is chosen from the group consisting of a preservative, in particular an antimicrobial agent or an antioxidant agent, a perfume, a masking agent, and mixtures thereof.

In the context of the invention, the term “preservative” refers to an agent that protects the composition according to the invention from changes in its physical or chemical state, its colour or its odour, or protects it from microbial contamination or degradation, for example by inhibiting the development of micro-organisms during the time the composition according to the invention is stored or used. Such an agent must be compatible with use of the composition according to the invention in contact with an animal or a human, and is advantageously non-irritating to the skin and eyes.

Examples of preservatives are known to the skilled person and include in particular benzyl alcohol, didecyldimonium chloride, caprylyl glycol, phenylpropanol, sodium benzoate, phenyl acetate, sorbic acid, dehydroacetic acid, potassium sorbate, phenoxyethanol, cetrimonium bromide, triclosan, parabens and thiazolinones.

In the context of the invention, the term “antimicrobial agent” therefore refers to a preservative that slows down the growth of micro-organisms (bacteria, fungi, yeasts, moulds) and combats the development of microbes in the composition according to the invention, when it is stored and/or when it is used.

Examples of antimicrobial agents are known to those skilled in the art and include in particular glycerin/phenyl alcohol/caprylhydroxamic acid, in particular marketed under the name Aromastat® by INOLEX.

The composition according to the invention may comprise from 0% to 4% by weight, preferably from 0.2% to 3.2% by weight, even more preferably 1% by weight of preservative, in particular antimicrobial agent, relative to the total weight of the composition according to the invention. In the context of the invention, the term “antioxidant agent” refers to a preservative capable of inhibiting reactions promoted by oxygen, thereby preventing oxidation.

Examples of antioxidants are known to those skilled in the art and include in particular compounds of the cinnamic acid family and their derivatives, such as pentaerythrityl tetra-di-t-butyl hydroxyhydrocinnamate in particular marketed under the name Tinogard TT® by BASF, vitamin A (retinol), vitamin C (ascorbic acid) and derivatives (ascorbate), vitamin E (tocopherol) and derivatives (tocopheryl), preferably Tinogard TT®.

The composition according to the invention may comprise from 0% to 2% by weight, preferably from 0.02% to 0.3% by weight, even more preferably 0.1% by weight of antioxidant agent, relative to the total weight of the composition.

In the context of the invention, the term “perfume” or perfuming agent refers to any hydrophobic substance designating a pure chemical or a mixture that is insoluble or sparingly soluble in water and capable of giving off a pleasant odour.

The composition according to the invention may comprise from 0% to 3% by weight, preferably from 0.2% to 2.5% by weight, even more preferably 2% by weight of perfume, relative to the total weight of the composition.

In the context of the invention, the term “masking agent” refers to an agent that reduces or inhibits the basic odour or taste of the product.

Examples of masking agents are known to the skilled person and include in particular beta sitosterol, citric acid, bisabolol and Calendula officinalis flower extract.

The composition according to the invention can comprise from 0% to 3% by weight, preferably from 0.2% to 2.5% by weight, even more preferably 1% or 2% by weight of a masking agent, relative to the total weight of the composition.

In a particular embodiment of the invention, said at least one additive is added to the oily phase, i.e., the oily phase further comprises at least one additive as defined above.

In another particular embodiment of the invention, said at least one additive, for example a perfume, is added after the formation of the oil-in-water emulsion, or after a step for adjusting the pH of the oil-in-water emulsion.

In a preferred embodiment of the invention, the oily phase also comprises at least one additive selected from the group consisting of a preservative, in particular an antimicrobial agent or an antioxidant agent, a perfume, a masking agent, and mixtures thereof, even more preferably at least one additive selected from the group consisting of a preservative, in particular an antimicrobial agent or an antioxidant agent, a masking agent, and mixtures thereof.

In another preferred embodiment of the invention, the composition according to the invention also comprises at least one additive such as a perfume. In particular, said perfume is added after the formation of the oil-in-water emulsion or after a step for adjusting the pH of the oil-in-water emulsion in order to avoid degradation of the perfume or loss of the perfume odour.

In a particular embodiment of the invention, the composition comprises from 60% to 95% by weight of water, preferably from 70% to 80% by weight of water, relative to the total weight of the composition, and/or comprises from 0.5% to 5% by weight, preferably from 1% to 2% by weight, of said semiochemical composition, relative to the total weight of the composition.

In a preferred embodiment of the invention, the composition comprises:
a) an aqueous phase comprising water, glycerol and xanthan gum,
b) an oily phase comprising at least one C₁₂-C₂₀ alkyl polyglucoside and at least one C₁₄-C₂₂ fatty alcohol (Montanov L® in particular marketed by SEPPIC), a mixture of myristic alcohol and myristylic glucoside (Montanov 14® in particular marketed by SEPPIC), caprylic/capric acid triglycerides (Myritol 318® in particular marketed by BASF), sunflower oil, glyceryl stearate (SustOleo GMS® in particular marketed by INOLEX), glycol palmitate (Lanol Pecailles® in particular marketed by SEPPIC), pentaerythrityl tetra-di-t-butyl hydroxyhydrocinnamate (Tinogard TT® in particular marketed by BASF), glycerin/phenyl alcohol/caprylhydroxamic acid (Aromastat® in particular marketed by INOLEX), optionally a perfume, and a semiochemical composition, the semiochemical composition being present in the composition according to the invention in an amount ranging from 0.5% to 5% by weight, preferably from 1% to 2% by weight, relative to the total weight of the O/W composition according to the invention.

In an even more preferred embodiment of the invention, the composition according to the invention comprises:
a) an aqueous phase comprising water in an amount ranging from 62% to 91% by weight, preferably from 73.0% to 76.5% by weight, even more preferably 74.4% or 76.1% by weight, glycerol in an amount ranging from 4.5% to 12% by weight, preferably 5% by weight, and xanthan gum in an amount ranging from 0.05% to 0.9% by weight, preferably from 0.2% to 0.4% by weight, even more preferably 0.2% by weight,
b) an oily phase comprising at least one C₁₂-C₂₀ alkyl polyglucoside and at least one C₁₄-C₂₂ fatty alcohol (Montanov L®, marketed in particular by SEPPIC) in an amount ranging from 1.3% to 8.5% by weight, preferably 4.5% by weight; a mixture of myristic alcohol and myristylic glucoside (Montanov 14®, in particular marketed by SEPPIC) in an amount ranging from 0.2% to 5.2% by weight, preferably 1% by weight; caprylic/capric acid triglycerides (Myritol 318®, in particular marketed by BASF) in an amount ranging from 0.5% to 8% by weight, preferably 3% by weight; sunflower oil in an amount ranging from 1% to 10.2% by weight, preferably 2% by weight; glyceryl stearate (SustOleo GMS®, in particular marketed by INOLEX) in an amount ranging from 0.2% to 5.2% by weight, preferably 2% by weight, and glycol palmitate (Lanol P ecailles®, in particular marketed by SEPPIC) in an amount ranging from 0.5% to 8.0% by weight, preferably 4.0% by weight; pentaerythrityl tetra-di-t-butyl hydroxyhydrocinnamate (Tinogard TT®, in particular marketed by BASF) in an amount ranging from 0.02% to 0.3% by weight, preferably 0.1% by weight, glycerin/phenyl alcohol/caprylhydroxamic acid (Aromastat®, in particular marketed by INOLEX) in an amount ranging from 0.2% to 3.2% by weight, preferably 1% by weight, and a semiochemical composition in an amount ranging from 0.5% to 5% by weight, preferably from 1% to 2% by weight, the amounts being expressed by weight relative to the total weight of the O/W composition according to the invention.

In a particular embodiment of the invention, the composition has a Brookfield viscosity greater than or equal to 5500 mPa·s, preferably 5500 mPa·s, as measured at 25° C. The viscosity within the meaning of the present invention is a Brookfield viscosity measured at room temperature (25° C.), preferably after 24 hours, by means of a viscometer, for example a Brookfield digital viscometer (model LDVI Prime), mobile S 63 and rotation speed 6 RPM.

In a particular embodiment of the invention, the composition in the form of an oil-in-water (O/W) emulsion has a pH of between 5 and 8, preferably a pH of between 6 and 7. Preferably, the composition has a pH value compatible with topical, in particular cutaneous, application of the composition to animal or human skin.

### Processes for Preparing the Composition of the Invention

The invention also relates to a first process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent, said semiochemical composition and optionally said at least one additive are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, a perfume is added;
f) the composition is recovered in the form of an oil-in-water (O/W) emulsion obtained in step c), d) or e).

The invention also relates to a second process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent and said semiochemical composition are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, said at least one additive is added;
f) the composition is recovered in the form of an oil-in-water (O/W) emulsion obtained in step c), d) or e).

Preferably, the invention relates to the first process for preparing a composition according to the invention.

The definitions and/or particular/preferred embodiments mentioned in the following paragraphs are applicable to both the first and second processes for preparing the composition according to the invention.

In the context of the invention, the term “pasting” means that before adding the water in step a) of the process, the at least one gelling agent is dispersed in the at least one humectant, in order to obtain a smooth, homogeneous paste. This pasting (or mashing) step, known to those skilled in the art, separates the particles and prevents the formation of lumps.

In a particular embodiment of the first or second process according to the invention, the pasting step a) is carried out at room temperature; and/or
the homogeneous aqueous phase obtained in step a) is kept under stirring at a temperature of between 65° C. and 75° C., preferably at a temperature of 70° C.; and/or
the homogeneous oily phase obtained in step b) is kept under stirring at a temperature of between 65° C. and 75° C., preferably at a temperature of 70° C.; and/or
step c) is carried out by mixing the oily phase from step b) with the aqueous phase from step a) at a temperature of between 65° C. and 75° C., preferably at a temperature of 70° C., and the emulsion is cooled to room temperature; and/or
step d) is carried out by adding a basic solution, for example a NaOH solution, in particular a 10% NaOH solution, in order to increase the pH of the emulsion obtained in step c), preferably in order to obtain a pH of between 5 and 8, preferably a pH of between 6 and 7.

In a particular embodiment of the process according to the invention, the aqueous phase obtained in step a) is in the form of a homogeneous mixture.

In a particular embodiment of the process according to the invention, the oily phase obtained in step b) is in the form of a homogeneous mixture.

In the context of the invention, the term “homogeneous mixture” refers to a mixture in which the individual constituents cannot be distinguished by the naked eye.

In a particular embodiment of the process according to the invention, the oil-in-water (O/W) emulsion obtained in step c) is a stable and homogeneous emulsion as defined above.

In a particular embodiment of the process according to the invention, step d) is obligatory for the preparation of the composition in the form of an oil-in-water (O/W) emulsion, comprising a semiochemical composition comprising (i) 3-cyclopentyl propionic acid and 3-cyclohexyl propionic acid, preferably 50% by weight of 3-cyclopentyl propionic acid and 50% by weight of 3-cyclohexyl propionic acid (CS1).

The invention relates in particular to a process for preparing the composition according to the invention, in particular a first or second process for preparing the composition according to the invention as described above, in which the compounds of the aqueous and oily phases are as described above.

It is also an object of the invention to use a composition consisting of an aqueous phase and a composition consisting of an oily phase as described above and exemplified below, to produce the composition according to the invention comprising, as active agent, a semiochemical composition as defined above, in the form of an oil-in-water (O/W) emulsion, in particular an oil-in-water emulsion which is stable and has a homogeneous appearance. In particular, the composition is in the form of a milk, preferably in the form of a white milk, having an opaque and thick texture (not flowing naturally out of the container in which it is contained).

### Non-Therapeutic Use of the Invention

According to another aspect, the invention relates to a non-therapeutic use of the composition according to the invention. According to this aspect, the composition according to the invention is used to obtain the semiochemical effect(s) expected from the semiochemical composition it contains.

Use of the composition according to the invention comprises topical, in particular cutaneous, administration of said composition to an animal, in particular a mammal, or to a human, or to animals treated as a group.

In a particular embodiment according to the invention, the animal is a domestic animal or a farm animal, in particular a mammal, preferably the animal is chosen from the group consisting of pets such as rabbits, cats, dogs, birds or from the group of farm animals such as horses or production animals such as pigs, cattle, poultry, for example chicken.

In a particular embodiment of the invention, the composition is administered topically, in particular cutaneously, preferably uniformly, to the skin of an animal or human. Preferably, the composition is applied uniformly to the part or parts of the skin of an animal or human where haematophagous invertebrates, in particular haematophagous insects such as mosquitoes, mites or ticks, could land, or to all the skin of an animal or human.

In particular, the invention relates to a non-therapeutic use of a composition in the form of an oil-in-water (O/W) emulsion according to the invention, for topical, in particular cutaneous, administration to an animal, in particular a mammal, or to a human, in particular to prevent invertebrates (haematophagous or not) from settling on the skin, or to prevent bites by haematophagous invertebrates, in particular haematophagous insects, such as mosquitoes, mites or ticks; or to prevent infestation by parasites such as poultry red mite (Dermanyssus sp. or Ornithonyssus sylviarum).

In a particular embodiment according to the invention, the composition comprises a semiochemical composition comprising from 40% to 60% of 3-cyclopentyl propionic acid and from 40% to 60% of 3-cyclohexyl propionic acid, preferably 50% of 3-cyclopentyl propionic acid and 50% of 3-cyclohexyl propionic acid; and relates to the non-therapeutic use of said composition for topical, in particular cutaneous, administration to an animal, in particular a mammal, or to a human, or to animals treated as a group, in particular to prevent invertebrates (haematophagous or not) from settling on the skin, or to prevent bites by haematophagous invertebrates, in particular haematophagous insects, such as mosquitoes, mites or ticks.

In a particular embodiment according to the invention, the composition comprises a semiochemical composition, in particular comprising an allomone produced by the uropygial glands of ducks, comprising from 40% to 60% by weight of bis(2-ethylhexyl) adipate and from 40% to 60% by weight of 1-isopropyl-2,2-dimethyltrimethylene diisobutyrate, preferably 50% by weight of bis(2-ethylhexyl) adipate and 50% by weight of 1-isopropyl-2,2-dimethyltrimethylene diisobutyrate; and relates to the non-therapeutic use of said composition for the topical, in particular cutaneous, administration to poultry such as hens, in particular on the skin of poultry such as a hen, in particular as a repellent allomone, in particular to prevent infestation by parasites such as the poultry red mite (Dermanyssus sp. or Ornithonyssus sylviarum). Another non-therapeutic use of said composition concerns the topical, in particular cutaneous, administration to an animal, for example birds or mammals, in particular on the skin of birds or mammals, or to a human, in particular on human skin, in particular to prevent infestation or stings by invertebrates, in particular haematophagous insects.

The above-mentioned use of said composition according to the invention is a non-therapeutic use, and excludes any therapeutic use practised on the animal or human body.

### EXAMPLES

The following examples are given by the way of illustration and therefore should not be construed to limit the scope of the invention.

### Example 1. Oil-in-Water Emulsions (Milk)

A composition in the form of an oil-in-water emulsion (milk) according to the invention having the composition shown in the following Table 1 was prepared.

TABLE 1

| | | | | Percentage |
|---|---|---|---|---|
| Component name | Function | INCI | CAS No. | by weight |
| Aqueous phase | | | | |
| Water | | AQUA | 7732-18-5 | qsp |
| Glycerol | Humectant | GLYCERIN | 56-81-5 | 5.0% |
| Xanthan gum | Gelling agent | XANTHAN GUM | 11138-66-2 | 0.2-0.4% |
| Oily phase | | | | |
| Sunflower oil | Emollient | HELIANTHUS ANNUUS | 8001-21-6 | 2.0%. |
| | agent | (SUNFLOWER) SEED OIL | | |
| Montanov L | Emulsifying | C14-22 ALCOHOLS & | | 4.5-5.0% |
| | agent | C12-20 ALKYL GLUCOSIDE | | |
| Montanov 14 | Emulsifying | MYRISTYL ALCOHOL & | 112-72-1 | 1.0% |
| | agent | MYRISTYL GLUCOSIDE | | |
| SustOleo GMS | Emulsifying | GLYCERYL STEARATE | 67701-33-1 | 1.0% |
| | agent | | | |
| Myritol 318 | Emollient | CAPRYLIC/CAPRIC | 73398-61-5 | 3.0% |
| | agent | TRIGLYCERIDE | | |
| Lanol P ecailles | Emulsifying | GLYCOL PALMITATE | 4219-49-2 | 4.0% |
| | agent | | | |
| Tinogard TT | Antioxidant | PENTAERYTHRITYL | 6683-19-8 | 0.1% |
| | agent | TETRA-DI-T-BUTYL | | |
| | | HYDROXYHYDRO- | | |
| | | CINNAMATE | | |
| Aromastat | Antimicrobial | GLYCERIN/PHENETHYL | 56-81-5 | 1.0% |
| | agent | ALCOHOL | 60-12-8 | |
| | | CAPRYLHYDROXAMIC | 7377-03-9 | |
| | | ACID | | |
| Perfume | Additive | / | / | 1.0% |
| Semiochemical | | / | / | 1%-1.98% |
| composition | | | | |
| (CS1 or CS2) | | | | |
| 10% NaOH | | SODIUM HYDROXIDE | 1310-73-2 | 0.06%-0.12% |
| solution | | | | |

### Semiochemical Composition Used as an Example in the Formulation of Table 1

CS1: 50% by weight of 3-cyclopentyl propionic acid and 50% by weight of 3-cyclohexyl propionic acid;

CS2: 50% by weight of bis(2-ethylhexyl) adipate and 50% by weight of 1-isopropyl-2,2-dimethyltrimethylene diisobutyrate.

### Example 2. Process for Preparing the Oil-in-Water Emulsion (Milk) According to Example 1

The oil-in-water emulsion (milk) according to Example 1 was prepared according to the following protocol:
STEP 1: Preparing the paste/aqueous phase
1. The glycerin and xanthan gum were weighed and added into a container adapted to contain the final volume of the aqueous phase. The mixture was stirred at room temperature.
2. Once the pasting was complete, all the water was added.
3. After homogenising the aqueous phase (obtaining a homogeneous mixture), the aqueous phase was kept at 70° C. and stirred until step 3.
STEP 2: Preparing the oily phase
4. The components of the oily phase, namely Sunflower oil, Montanov L, Montanov 14, SustOleo GMS, Myritol 318, Lanol P ecailles, Tinogard TT, Aromastat and the semiochemical composition, were weighed and added to another container suitable for containing the final volume of the oily phase.
5. Once all the compounds had been added, the mixture was stirred and heated to 70° C. until a homogeneous mixture was obtained.
STEP 3: Formation of the oil-in-water emulsion
6. When the two phases (aqueous and oily) were perfectly homogeneous, the oily phase was added at 70° C. to the aqueous phase at 70° C. Care was taken to ensure that the emulsion was well agitated throughout this stage. The emulsion was then cooled to room temperature after homogenising the emulsion.
STEP 4: Modification of the pH only for the CS1 composition mentioned above
7. To increase the pH, a pH adjustment step was carried out by adding a 10% NaOH solution until a pH of between 6 and 7 was obtained.
STEP 5: Perfume
8. Add any perfume with stirring at room temperature
STEP 6: Recovery of the oil-in-water (O/W) emulsion

The final emulsion obtained in the form of a milk was white, opaque and thick.

### Example 3. Organoleptic Measurements of the Oil-in-Water Emulsions (Milk) According to Example 1

4 criteria (appearance, colour, smell, texture) were selected for organoleptic measurements.
Appearance: Thick milk
Colour: White
Odour: Characteristic odour
Texture: light texture (fluid) and rich texture (cream)

### Example 4. pH measurements of the Oil-in-Water Emulsion (Milk) According to Example 1

The pH measurements were carried out using a LE 438 IP 67 electrode marketed by Mettler Toledo®. This is a 2-in-1 glass pH electrode with integrated temperature sensor and liquid electrolyte for rapid measurements in the laboratory of aqueous samples such as acids, bases, etc.

The specifications for this electrode are given in Table 2 below.

TABLE 2

| | |
|---|---|
| Measuring range | pH 0-14 |
| Zero point | 7.00 ± 0.25 |
| Temperature range | 0-80° C. |
| Temperature sensor | NTC 30 K Ω |
| Membrane resistance | <250 MΩ |
| (25° C.) | |
| Reference electrolyte | 3 mol/L KCl solution |
| | saturated with AgCl |
| Reference system | Ag/AgCl |
| Reference diaphragm | Gel |

Before any measurements are taken, the pH meter used is calibrated. The nature of the semiochemical composition can affect the pH of the emulsion, as some semiochemicals have an acid pH. For topical application, particularly cutaneous, it is therefore necessary to measure the pH of each emulsion obtained and, if necessary, adjust the pH to obtain a pH of between 5 and 8, preferably between 6 and 7.

A pH measurement was carried out after the emulsion was obtained, and is reported below:
MILK with CS1:4.0<pH<5.0
MILK with CS2:5.5<pH<7.5

In order to be as close as possible to the pH of animal or human skin, and to avoid causing an irritation reaction of the skin, the pH of the formulation corresponding to MILK with CS1 has been adjusted.

Following this adjustment, a new pH measurement was carried out and is reported below:
MILK with CS1:6.0<pH<7.0

For the formulation corresponding to the MILK with CS2, no pH adjustment step was carried out.

### Example 5. Measurement of the Viscosity of the Oil-in-Water Emulsion (Milk) According to Example 1

Viscosity is the resistance of a fluid to the sliding of one layer onto another. For each formulation, the viscosity must be checked after each production run. Viscosity is influenced by a number of factors, including temperature, the measurement device used, the measurement speed used and the time of measurement during production (measurements at T0 and T+24h, T+48h). The viscosity should be measured when the solution is at room temperature, preferably at T+24h.

The viscosity of the oil-in-water emulsion according to Example 1 was measured using a Brookfield digital viscometer (model DV-I Prime), mobile S 63 and at a rotation speed of 6 RPM.

The Brookfield DV-I Prime viscometer measures the viscosity of fluids at a given velocity gradient. The principle of operation involves rotating a moving part (which is immersed in the liquid to be tested) by means of a calibrated spring. The measurement range (in centipoises or milliPascal-seconds) is determined by the speed of rotation of the mobile, the size and shape of the mobile, the cross-section where the mobile is rotating and the full scale of the torsional moment of the calibrated spring.

The specifications of the Brookfield DV-I Prime viscometer are given in Table 3 below.

TABLE 3

| | |
|---|---|
| Speeds (rpm) | 0.0; 0.3; 0.6; 1.5; 3; 6; 12; 30; 60; |
| | 0.5; 1; 2; 2.5; 4; 5; 10; 20; 50; 100 |
| Viscosity range (cP) | 15/2.000.000 |
| Operating medium | Temperature range 0° C. to 40° C. |
| | (32° F. to 104° F.) 20% to 80% |
| | R.H. non-condensing |
| Analogous torque output | 0-1 Vdc (torque from 0 to 100%) |
| Accuracy of viscosity | ±1° C.: - of full scale |
| measurement | |
| Reproducibility of | 0.2% of full scale used |
| viscosity measurement | |
| Devices used | S61; S62; S63; S64 |

As a result of these measurements, all the oil-in-water emulsions (milk) according to Example 1 had a viscosity>5500 cP or mPa·s.

### REFERENCES

Silverstein, R.M., 1977. “Complexity, diversity and specificity of behaviour-modifying chemicals; examples from Coleoptera and Hymenoptera,” Chemical Control of Insect Behavior: Theory and Application. Publishers H.H. Shorey & J.J.Mckelvey pp.231-251 New York: Plenum Press);
Wyatt T.D., 2003 “Pheromones and Animal Behavior. Communication by smell and taste, pp. 1-4 New York: Cambridge University Press.

1. A composition comprising:
a) an aqueous phase comprising water, at least one humectant chosen from polyols and sugars, and at least one gelling agent chosen from polysaccharides,
b) an oily phase comprising at least one emollient agent, at least one emulsifying agent, and a semiochemical composition,
wherein the semiochemical composition comprises semiochemical compounds selected from the group consisting of:
(i) a mixture of 3-cyclopentyl propionic acid and 3-cyclohexyl propionic acid, and
(ii) a mixture of bis(2-ethylhexyl) adipate and 1-isopropyl-2,2-dimethyltrimethylene diisobutyrate;
wherein the composition is in the form of an oil-in-water (O/W) emulsion.

2. The composition according to claim 1, wherein the semiochemical composition comprises:
(i) from 40% to 60% by weight of 3-cyclopentyl propionic acid, and from 40% to 60% by weight of 3-cyclohexyl propionic acid, or
(ii) from 40% to 60% by weight of bis(2-ethylhexyl) adipate, and from 40% to 60% by weight of 1-isopropyl-2,2-dimethyltrimethylene diisobutyrate,
the total sum of the semiochemical compounds present in the semiochemical composition being equal to 100%.

3. The composition according to claim 2, wherein the semiochemical composition comprises:
(i) 50% by weight of 3-cyclopentyl propionic acid, and 50% by weight of 3-cyclohexyl propionic acid, or
(ii) 50% by weight of bis(2-ethylhexyl) adipate, and 50% by weight of 1-isopropyl-2,2-dimethyltrimethylene diisobutyrate.

4. The composition according to claim 1, wherein the at least one humectant is chosen from polyols selected from the group consisting of glycerol, propylene glycol, polyethylene glycols (PEG), xylitol, isomalt, maltitol, mannitol, polydextrose, sorbitol, isomaltitol, lactitol, and mixtures thereof; and/or from sugars selected from the group consisting of fructose, glucose and lactose.

5. The composition according to claim 4, wherein the at least one humectant is glycerol.

6. The composition according to claim 1, wherein the at least one humectant is present in an amount ranging from 4% to 12% by weight, relative to the total weight of the composition.

7. The composition according to claim 1, wherein the polysaccharides are chosen from the group consisting of xanthan gum, guar gum, cellulose, cellulose derivatives, maltodextrin, maltodextrin derivatives and mixtures thereof.

8. The composition according to claim 7, wherein the polysaccharides are xanthan gum.

9. The composition according to claim 1, wherein the polysaccharides are present in an amount ranging from 0.05% to 1% by weight, relative to the total weight of the composition.

10. The composition according to claim 1, which comprises from 60% to 95% by weight of water, relative to the total weight of the composition, and/or from 0.5% to 5% by weight, of the semiochemical composition, relative to the total weight of the composition.

11. The composition according to claim 1, wherein the at least one emollient agent is selected from the group consisting of hydrogenated vegetable oils, non-hydrogenated vegetable oils, vegetable oils comprising triglycerides of unsaturated fatty acids, silicone oils, mineral oils, waxes, fatty alcohols, fatty acids, fatty acid esters, glyceride derivatives, glycerol derivatives, and mixtures thereof.

12. The composition according to claim 11, wherein the at least one emollient agent is a mixture of emollients consisting of glyceride derivatives and vegetable oils comprising triglycerides of unsaturated fatty acids.

13. The composition according to claim 1, wherein the at least one emollient agent is present in an amount ranging from 1% to 19% by weight, relative to the total weight of the composition.

14. The composition according to claim 1, wherein the at least one emulsifying agent is selected from the group consisting of alkyl polyglucosides, mixtures of at least one alkyl polyglucoside and at least one fatty alcohol, fatty acid esters, and fatty acid alcohol esters.

15. The composition according to claim 1, wherein the at least one emulsifying agent is present in an amount ranging from 2% to 30% by weight, relative to the total weight of the composition.

16. The composition according to claim 1, wherein the oily phase further comprises at least one additive chosen from the group consisting of a preservative, a perfume, a masking agent, and mixtures thereof.

17. The composition according to claim 1, which is in the form of a milk.

18. The composition according to claim 1, wherein it comprises:
a) an aqueous phase comprising water, glycerol and xanthan gum,
b) an oily phase comprising at least one C12-C20 alkyl polyglucoside and at least one C₁₄-C₂₂ fatty alcohol, a mixture of myristic alcohol and myristylic glucoside, caprylic/capric acid triglycerides, sunflower oil, glyceryl stearate, glycol palmitate, pentaerythrityl tetra-di-t-butyl hydroxyhydrocinnamate, glycerol/phenyl alcohol/caprylhydroxamic acid, and a semiochemical composition,
the semiochemical composition being present in the composition in the form of an O/W emulsion in an amount ranging from 0.5% to 5% by weight, relative to the total weight of the composition.

19. A process for preparing the composition in the form of an oil-in-water (O/W) emulsion according to claim 1, comprising the following steps:
a) the at least one gelling agent is pasted into the at least one humectant; then water is added with stirring and at a temperature of between 65° C. and 75° C., so as to obtain a homogeneous aqueous phase;
b) the at least one emollient agent, the at least one emulsifying agent, the semiochemical composition and optionally the at least one additive are mixed, with stirring and at a temperature of between 65° C. and 75° C., so as to obtain a homogeneous oily phase;
c) the oily phase from step b) is added, with stirring, to the aqueous phase from step a) so as to obtain an O/W emulsion;
d) optionally, the pH of the emulsion obtained in step c) is adjusted;
e) optionally, a perfume is added;
f) the composition is recovered in the form of an O/W emulsion obtained in step c), d) or e).

20. The process according to claim 19, wherein:
the pasting step a) is carried out at room temperature; and/or
the homogeneous aqueous phase obtained in step a) is kept under stirring at a temperature of between 65° C. and 75° C.; and/or
the homogeneous oily phase obtained in step b) is kept under stirring at a temperature of between 65° C. and 75° C.; and/or
step c) is carried out by mixing the oily phase from step b) with the aqueous phase from step a) at a temperature of between 65° C. and 75° C., and the emulsion is cooled to room temperature; and/or
step d) is carried out by adding a basic solution.

21. Non-therapeutic use of the composition in the form of an oil-in-water (O/W) emulsion according to claim 1, wherein the use comprises topical, administration of the composition according to claim 1 to an animal or to a human, to prevent invertebrates from settling on the skin, or to prevent bites by haematophagous invertebrates; or to prevent infestation by parasites.

22. The non-therapeutic use according to claim 21, wherein the haematophagous invertebrates are selected from the group consisting of mosquitoes, mites and ticks.

23. The non-therapeutic use according to claim 21, wherein the parasites are poultry red mite (Dermanyssus sp. or Ornithonyssus sylviarum).
